(19) United States
(12) Patent Application Publication
(10) Pub. No.: US 2023/0355205 A1
(43) Pub. Date: Nov. 9, 2023

(54) ADDITION OF A PRESSURE/FORCE SENSOR TO AN ULTRASOUND PROBE

(71) Applicant: University of South Carolina, Columbia, SC (US)

(72) Inventor: Richard Hoppmann, Columbia, SC (US)

(73) Assignee: University of South Carolina, Columbia, SC (US)

(21) Appl. No.: 18/313,591

(22) Filed: May 8, 2023

Related U.S. Application Data
(60) Provisional application No. 63/339,549, filed on May 9, 2022.

(51) Int. Cl. A61B 8/00

(52) U.S. Cl. CPC A61B 8/4444; A61B 8/429; A61B 8/4254; A61B 8/461

(57) ABSTRACT

Described herein are diagnostic methods and systems employing a sensor added to an ultrasound probe to measure the pressure/force being applied to the patient by the probe during ultrasound scanning, feedback of the probe contact pressure measurement will provide sonographers an opportunity to make probe pressure adjustments to both capture a quality image and minimize discomfort to the patient, as well as enhance the diagnostic capability of other forms of ultrasound.

### TECHNICAL FIELD

The subject matter disclosed herein is generally directed to diagnostic methods and systems employing a sensor added to an ultrasound probe to measure the pressure/force being applied to the patient by the probe during ultrasound scanning, feedback of the probe contact pressure measurement will provide sonographers an opportunity to make probe pressure adjustments to both capture a quality image and minimize discomfort to the patient, as well as enhance the diagnostic capability of other forms of ultrasound.

### BACKGROUND

Medical ultrasound falls into two distinct categories: diagnostic and therapeutic. Diagnostic ultrasound is a non-invasive diagnostic technique used to image inside the body. Ultrasound probes, called transducers, produce sound waves that have frequencies above the threshold of human hearing (above 20 KHz), but most transducers in current use operate at much higher frequencies (in the megahertz (MHz) range). Most diagnostic ultrasound probes are placed on the skin. However, to optimize image quality, probes may be placed inside the body via the gastrointestinal tract, vagina, or blood vessels. In addition, ultrasound is sometimes used during surgery by placing a sterile probe into the area being operated on.

Diagnostic ultrasound can be further sub-divided into anatomical and functional ultrasound. Anatomical ultrasound produces images of internal organs or other structures. Functional ultrasound combines information such as the movement and velocity of tissue or blood, softness or hardness of tissue, and other physical characteristics, with anatomical images to create “information maps.” These maps help doctors visualize changes/differences in function within a structure or organ.

Therapeutic ultrasound also uses sound waves above the range of human hearing but does not produce images. Its purpose is to interact with tissues in the body such that they are either modified or destroyed. Among the modifications possible are: moving or pushing tissue, heating tissue, dissolving blood clots, or delivering drugs to specific locations in the body. These destructive, or ablative, functions are made possible by use of very high-intensity beams that can destroy diseased or abnormal tissues such as tumors. The advantage of using ultrasound therapies is that, in most cases, they are non-invasive. No incisions or cuts need to be made to the skin, leaving no wounds or scars.

Ultrasound waves are produced by a transducer, which can both emit ultrasound waves, as well as detect the ultrasound echoes reflected back. In most cases, the active elements in ultrasound transducers are made of special ceramic crystal materials called piezoelectrics. These materials are able to produce sound waves when an electric field is applied to them, but can also work in reverse, producing an electric field when a sound wave hits them. When used in an ultrasound scanner, the transducer sends out a beam of sound waves into the body. The sound waves are reflected back to the transducer by boundaries between tissues in the path of the beam (e.g. the boundary between fluid and soft tissue or tissue and bone). When these echoes hit the transducer, they generate electrical signals that are sent to the ultrasound scanner. Using the speed of sound and the time of each echo's return, the scanner calculates the distance from the transducer to the tissue boundary. These distances are then used to generate two-dimensional images of tissues and organs. During an ultrasound exam, the technician will apply a gel to the skin. This keeps air pockets from forming between the transducer and the skin, which can block ultrasound waves from passing into the body.

Diagnostic ultrasound is able to non-invasively image internal organs within the body. However, it is not good for imaging bones or any tissues that contain air, like the lungs. Under some conditions, ultrasound can image bones (such as in a fetus or in small babies) or the lungs and lining around the lungs, when they are filled or partially filled with fluid. One of the most common uses of ultrasound is during pregnancy, to monitor the growth and development of the fetus, but there are many other uses, including imaging the heart, blood vessels, eyes, thyroid, brain, breast, abdominal organs, skin, and muscles. Ultrasound images are displayed in either 2D, 3D, or 4D (which is 3D in motion).

Functional ultrasound applications include Doppler and color Doppler ultrasound for measuring and visualizing blood flow in vessels within the body or in the heart. It can also measure the speed of the blood flow and direction of movement. This is done using color-coded maps called color Doppler imaging. Doppler ultrasound is commonly used to determine whether plaque build-up inside the carotid arteries is blocking blood flow to the brain.

Another functional form of ultrasound is elastography, a method for measuring and displaying the relative stiffness of tissues, which can be used to differentiate tumors from healthy tissue. This information can be displayed as either color-coded maps of the relative stiffness; black- and white maps that display high-contrast images of tumors compared with anatomical images; or color-coded maps that are overlaid on the anatomical image. Elastography can be used to test for liver fibrosis, a condition in which excessive scar tissue builds up in the liver due to inflammation.

Ultrasound is also an important method for imaging interventions in the body. For example, ultrasound-guided needle biopsy helps physicians see the position of a needle while it is being guided to a selected target, such as a mass or a tumor in the breast. Also, ultrasound is used for real-time imaging of the location of the tip of a catheter as it is inserted in a blood vessel and guided along the length of the vessel. It can also be used for minimally invasive surgery to guide the surgeon with real-time images of the inside of the body.

Therapeutic ultrasound produces high levels of acoustic output that can be focused on specific targets for the purpose of heating, ablating, or breaking up tissue. One type of therapeutic ultrasound uses high-intensity beams of sound that are highly targeted, and is called High Intensity Focused Ultrasound (HIFU). HIFU is being investigated as a method for modifying or destroying diseased or abnormal tissues inside the body (e.g. tumors) without having to open or tear the skin or cause damage to the surrounding tissue. Either ultrasound or MRI is used to identify and target the tissue to be treated, guide and control the treatment in real time, and confirm the effectiveness of the treatment. HIFU is currently FDA approved for the treatment of uterine fibroids, to alleviate pain from bone metastases, and most recently for the ablation of prostate tissue. HIFU is also being investigated as a way to close wounds and stop bleeding, to break up clots in blood vessels, and to temporarily open the blood brain barrier so that medications can pass through.

An area in ultrasonography that has not been adequately addressed from either a practical ultrasound scanning perspective or clinical impact perspective has been the associated pressure or force of the probe on the patient produced by the ultrasound practitioner when scanning. The amount of pressure applied with the probe can affect the quality of image acquired because, in general, the closer one can get the probe to the target anatomical structure the better the image. To get closer to the structure requires additional probe pressure to penetrate more deeply into the body. For example, applying additional pressure with the probe on the abdomen will get the probe closer to the abdominal aorta and improve image quality of the aorta. Applying more probe pressure also helps move bowel gas from between the ultrasound probe and the aorta. Bowel gas prevents the ultrasound waves from uniformly reaching the aorta and reflecting back to the probe for image display. Moving gas out of the ultrasound wave path can markedly improve the quality of the image. Accordingly, it is an object of the present disclosure to provide improved methods, systems, and ultrasound probes employing a pressure sensor to further enhance ultrasound diagnostic and treatment regimens as well as ultrasound education.

Citation or identification of any document in this application is not an admission that such a document is available as prior art to the present disclosure.

### SUMMARY

The above objectives are accomplished according to the present disclosure by providing in one embodiment, an improved ultrasound probe. The probe may include at least one ultrasound probe comprising at least one pressure sensor, wherein the at least one pressure sensor is configured to measure pressure applied to a patient via the at least one ultrasound probe during ultrasound scanning; and at least one color-coded feedback display configured to display different colors correlating to different pressure levels. Further, the at least one ultrasound probe may be configured to display pressure measurements for an entirety of the at least one ultrasound probe. Still further, the at least one ultrasound probe may display differential measurements for at least one section of the at least one ultrasound probe. Further again, the at least one ultrasound probe may provide connectivity via cable connectivity, wireless, connectivity, or a combination of connectivity methods. Even further, the probe may include a probe contact configured to provide feedback regarding probe pressure applied to the patient.

In a further embodiment, the disclosure provides an improved system for conducting ultrasound scans. The system may include at least one ultrasound probe comprising at least one pressure sensor and wherein the at least one pressure sensor is configured to measure pressure applied to a patient via the at least one ultrasound probe during ultrasound scanning. Again further, the system may configure the at least one ultrasound probe to display pressure measurements for an entirety of the at least one ultrasound probe. Further still, the at least one ultrasound probe may be configured to display differential measurements for at least one section of the at least one ultrasound probe. Even further, the at least one ultrasound probe may provide connectivity via cable connectivity, wireless, connectivity, or a combination of connectivity methods. Still yet, the system may include a probe contact configured to provide feedback regarding probe pressure applied to the patient. Further again, the system may include at least one color-coded feedback display configured to display different colors correlating to a different pressure levels of the at least one ultrasound probe.

The disclosure may also provide an improved ultrasound diagnostic method. The method may include incorporating at least one pressure sensor into at least one ultrasound probe and the at least one pressure sensor may be configured to measure pressure applied to a patient via the at least one ultrasound probe during ultrasound scanning and the method may incorporate at least one color-coded feedback display configured to display different colors correlating to different pressure levels. Further again, the method may include increasing or reducing pressure of the at least one ultrasound probe in response to a pain indication from a patient. Yet further, the method may include increasing or reducing pressure of the at least one ultrasound probe to improve image quality of the at least one ultrasound probe. Yet still further, the method may include configuring the at least one ultrasound probe to display pressure measurements for an entirety of the at least one ultrasound probe. Moreover, the method may include configuring the at least one ultrasound probe to display differential measurements for at least one section of the at least one ultrasound probe. Furthermore, the method may configure the at least one ultrasound probe to provide connectivity via cable connectivity, wireless, connectivity, or a combination of connectivity methods. Again moreover, the method may include a probe contact configured to provide feedback regarding probe pressure applied to the patient.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows a traditional linear ultrasound probe with its probe surface: (a) uncompressed; and (b) compressed.

FIG. 2 shows a traditional liner ultrasound probe as well as three probes of the current disclosure.

FIG. 3 shows at: (a) a photograph of a traditional linear probe analyzing the thyroid gland of a patient; and (b) the ultrasound image obtained via the probe.

FIG. 4 shows at: (a) a probe of the current disclosure with uniform, moderate pressure; and (b) colored ultrasound images showing pressure displayed on the tissue visible via the ultrasound probe.

FIG. 5 shows at: (a) a probe of the current disclosure displaying a pressure gradient; and (b) the tissue visualized via the ultrasound probe also showing a color-coded pressure gradient.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

### Kits

Any of the systems, methods or probes described herein can be presented as a combination kit. As used herein, the terms “combination kit” or “kit of parts” refers to the systems, methods or probes and any additional components that are used to package, sell, market, deliver, and/or administer the combination of elements or a single element, such as the ultrasound probes described herein. Such additional components include, but are not limited to, packaging, blister packages, probes, sensor housings, and the like. When one or more of the systems, methods or probes, described herein or a combination thereof (e.g., a probe or system contained in the kit provided simultaneously, the combination kit can contain the systems, methods or probes in a single package or in separate formulations. When the systems, methods or probes described herein or a combination thereof and/or kit components are not administered simultaneously, the combination kit can contain each system, method or probe. The separate kit components can be contained in a single package or in separate packages within the kit.

In some embodiments, the combination kit also includes instructions printed on or otherwise contained in a tangible medium of expression. The instructions can provide information regarding the systems, methods or probes, safety information regarding the systems, methods or probes, information regarding, indications for use, and/or recommended treatment regimen(s) for the systems, methods or probes contained therein. In some embodiments, the instructions can provide directions and protocols for administering the systems, methods or probes described herein to a subject in need thereof. In some embodiments, the instructions can provide one or more embodiments of the methods for administration of the systems, methods or probes such as any of the methods described in greater detail elsewhere herein.

Medical ultrasound has grown significantly over the past two decades with tremendous advances in the technology and miniaturization of ultrasound devices that include powerful laptop-sized and hand-held ultrasound systems. There are many new clinical ultrasound applications and a rapidly expanding number of ultrasound users from primary care physicians to medical sub-specialists, nurses, nurse practitioners, physician assistants, and medical technicians. Ultrasound is considered an operator dependent imaging modality and requires significant knowledge and skill by the user as opposed to x-ray, computer tomography, and magnetic resonance imaging which are automated modalities with standard imaging protocols.

An area in ultrasonography that has not been adequately addressed from either a practical ultrasound scanning perspective or clinical impact perspective has been the associated pressure or force of the probe on the patient produced by the ultrasound practitioner when scanning. The amount of pressure applied with the probe can affect the quality of image acquired because, in general, the closer one can get the probe to the target anatomical structure the better the image. To get closer to the structure requires additional probe pressure to penetrate more deeply into the body. For example, applying additional pressure with the probe on the abdomen will get the probe closer to the abdominal aorta and improve image quality of the aorta. Applying more probe pressure also helps move bowel gas from between the ultrasound probe and the aorta. Bowel gas prevents the ultrasound waves from uniformly reaching the aorta and reflecting back to the probe for image display. Moving gas out of the ultrasound wave path can markedly improve the quality of the image.

Those new to ultrasound must learn to apply an appropriate amount of probe pressure to acquire a good image, but not too much pressure to cause unnecessary discomfort or pain to the patient. At present there is no objective quantitative feedback to the ultrasound user as to degree of pressure being applied. This invention would provide real-time digital feedback to the user on the amount of probe pressure being applied to the body surface. This method of feedback will help the inexperienced ultrasound user develop an appreciation of the proprioceptive sensation of producing various probe pressures and learn a range of appropriate probe pressures for various ultrasound applications.

There are also clinical scenarios when pressure with the ultrasound probe will elicit a pain response indicating possible pathology under the area being compressed with the probe. Such examples include pain over a gallbladder with cholecystitis, pain over a muscle area that is torn or inflamed, and painful trigger points of fibromyalgia or myofascial pain syndrome. In these cases, the quantitative measure of pressure applied over the painful area versus a contiguous non-painful area can be recorded and become an important clinical finding of the examination. Such information can also serve as a baseline to assess improvement or progression of disease over time and response to therapy. In addition, binary pain mapping can be performed by sweeping the probe over an area of interest and having the patient respond that the probe pressure is painful or not painful. This approach would assist in determining the existence and extent of painful areas in particular anatomical regions that might require further assessment and documentation.

Another valuable use of probe pressure measurement comes with the rapidly advancing technology of ultrasound elastography. Elastography is an imaging technology sensitive to tissue stiffness first described in the 1990s. It can qualitatively and quantitatively assess tissue stiffness. There are presently several elastography methods, including compression elastography, transient elastography, tension elastography, and shear-wave elastography. All of these methods take advantage of changes in the elasticity of tissue to help identify pathological processes. Common examples include the use of elastography to identify the increased stiffness of fibrosis in chronic liver disease and assessment of soft tissue stiffness in breast and thyroid lesions that can indicate malignancy. The degree of compression of the structure or more selectively the region of interest in the tissue by the ultrasound probe pressure can be incorporated into estimates of elastography.

In other forms of elastography such as shear-wave elastography probe pressures by the sonographer, especially if variable during measurements, can alter elastography measurements and their clinical value. The compressive force applied at the skin surface of the body diminishes progressively as a function of the depth and tissue composition of the structures encountered. However, for relatively superficial structures such as the thyroid gland, breast tissue, the globe of the eye, cutaneous and subcutaneous structures, muscles, tendons, and peripheral nerves compressive forces from the probe will provide valuable diagnostic and therapeutic clinical information. These objective clinical data points can also be used in artificial intelligence applications as additional data points in diagnostic assessment of pain, progression of pain, location of pain, and other clinical parameters of disease. Having pressure/force measurements coming directly from the probe surface itself as opposed to force measurements from a mechanical arm or hand will also advance ultrasound robotics.

Thus, having an objective quantitative measure of probe pressure levels can assist with learning appropriate pressure levels for various ultrasound applications, can be used to provide additional clinical data points such as more precise location and pressure thresholds for many clinical scenarios involving pain, and can enhance standardization and accuracy of various methods of ultrasound assessment with elastography as well as ultrasound robotics.

A number of approaches to measuring the amount of force being applied to the body surface are possible such as the traditional platform force and strain gauge methods as well as newer methods of micro-fabricated tactile sensors. The more traditional methods to measure pressure/force have been in use medically for many years to measure pain thresholds in various conditions including neuromuscular diseases, inflammatory diseases, trauma, and myofascial pain syndromes such as fibromyalgia and temporal mandibular syndrome.

Commercial traditional method devices are available that use pressure force measurements to assess soft tissue tone and pain. These include the tonometer which is a hand-held mechanical device to measure muscle tone. The tonometer can be used to assess muscle characteristics of healthy subjects as well as patients with various connective tissue and neurological diseases affecting muscle tone or soft tissue consistency. It can also detect muscle tone changes related to anesthetics, nerve blocks, muscle relaxants, and other medications. The body surface contact end of the device has a small flat surface area which is pressed against the body over an underlying muscle. The amount of force applied to the surface area is typically measured by a strain gauge. This process can be performed manually by an examiner or with computerized automation with force applied and distance traveled (tissue indentation) measured, recorded, and plotted graphically.

A similar device, called a pressure algometer, can be used to measure pain thresholds over soft tissue. These are typically handheld devices with a pressure gauge which can be digital and easy for the examiner to read denoting the force applied that induces pain as reported by the patient. Pain has historically been assessed by patient self-report using visual analog pain scales which are subjective and subject to over-estimation of pain by the patient. During the physical examination, the clinician can palpate the soft tissue and ask the patient if the examination is painful and how painful. The clinician can also observe the patient for physical responses to painful palpation such as wincing or withdrawing from the clinician's hand. It has been demonstrated that the degree of force applied by different examiners is quite variable and not reliably reproduced from exam to exam.

Because of issues with manual palpation, a similar device to the algometer called a palpometer can be used which has a small contact surface and a spring coil that can be set to produce a consistent force. Studies have shown that such a device is reliable and provides a more accurate and reproducible pressure stimulus than manual subjective hand palpation by the examiner.

The new micro-fabricated sensor methods commonly use piezoresistive, piezoelective, and capacitive sensors in a layered design to accurately measure force. These micro-electro-mechanical systems (EMMS), as well as nano-electro-mechanical systems (NENS) offer a variety of mechanisms to create ultrasound probes capable of accurate and clinically valuable pressure/force measurements. Flexforce is a piezoresistive sensor by Tekscan that is widely used in robotic hands and can precisely measure a range of force between any two surfaces.

Medical applications as they relate to pressure and force include use in prosthetics, tactile sensing for robotic surgery, and smart interfaces for biomedical measurements. These sensors have played a major role in the advancement of precise measurement and control of the tactile pressure/force needed in the robotics industry. They have the capability to measure the magnitude and direction of applied force, measure point of application of force on a contact surface and assess compliance and textual properties of objects manipulated. These properties of micro-fabricated methods, in addition to their accuracy and sensitivity in measurements, their small size, thin construction, flexibility, and the ability to embed them into a variety of device materials, make them very attractive as sensors for ultrasound probe pressure applications.

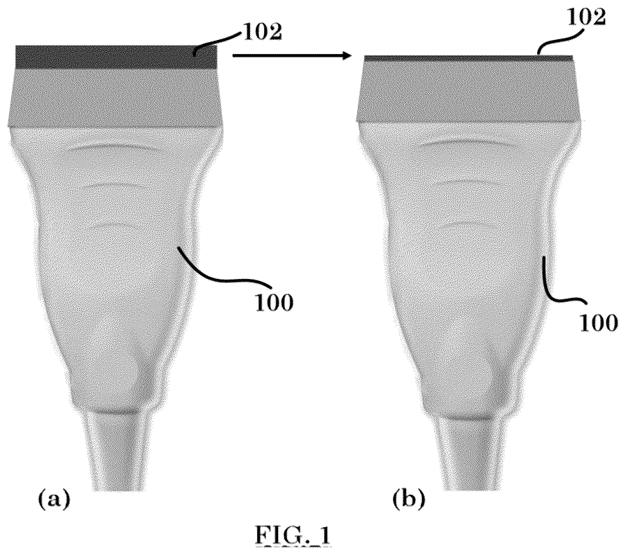

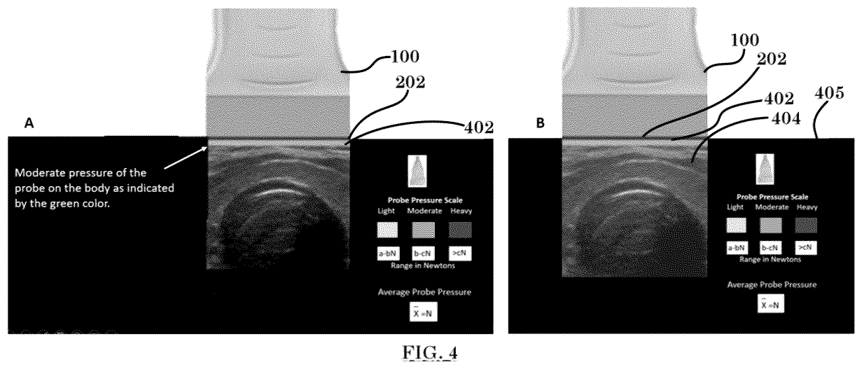

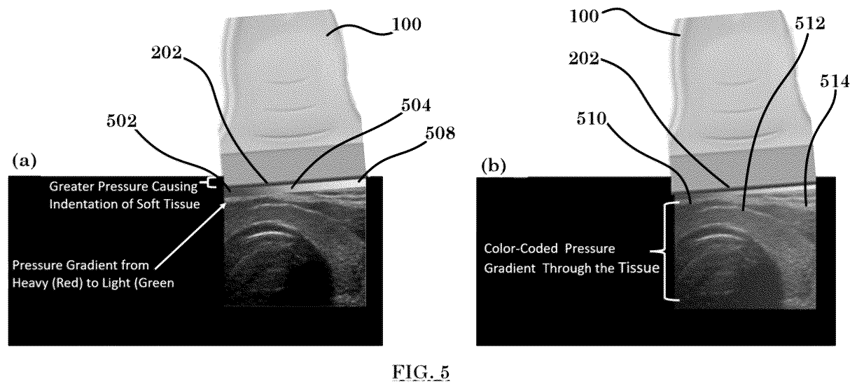

Probe pressure sensor arrays can be placed on the face of the ultrasound probe across the entire surface provided the material does not critically interfere with the ultrasound waves. Otherwise, the sensors material can be incorporated along the outer margin of the surface support material. A traditional pressure method approach or the micro-fabricated tactile sensor approach can be used with sensors embedded into compatible margin support material (see FIGS. 1-3). Sensor array companies are presently producing sensor arrays with a variety of sensor technologies, such as capacitance, micro-sensor size in millimeters, broad pressure ranges, pressure sensitivity about 0.05 kPa, and hysteresis of less than 4%. These specifications would be appropriate for use in a wide variety of ultrasound probes in use today. In addition to a single measure of probe pressures against the body surface, multiple individual sensors can be placed across the probe surface that can measure differential pressures at various locations of the probe (see FIGS. 4-5). Thus, measurement of pressure at the ends of the probe, the center of the probe, or across the entire probe can be measured. This multi-axial pressure sensing approach would be particularly important for non-linear probes such as the curvilinear ultrasound probe used primarily for scanning of the abdomen and pelvis.

These pressure forces can be displayed as numerical values as well as be color coded for the degree of pressure at that area of the probe (i.e., red for heavy pressure, green for moderate pressure, yellow for light pressure) and overlaid on the corresponding ultrasound image. This would be on a screen display in real time as the sonographer is scanning. This feature would also serve as a valuable teaching tool to demonstrate and give feedback to the learner in terms of how changes in pressure of the probe at different locations of the probe can affect the ultrasound image.

The current disclosure can be applied to all types of ultrasound probes and include all forms of probe-display system connectivity including cable connectivity, wireless connectivity (i.e., Bluetooth), or a combination of connectivity methods.

There are standard ultrasound probe maneuvers, such as the “heel-toe” maneuver, that requires heavy probe pressure to be shifted from one end of the probe (heel) across the probe to the other end (toe). This maneuver can help the operator identify the pressure distribution across the probe surface that results in the best image. Changing the pressure at one end of the probe will also change the angle of the probe resulting in a slightly different image displayed. Understanding this maneuver will be enhanced by the learner noting how differential pressure along the probe produces a slightly different image and allows the operator to capture the best image for the clinical examination. From an ultrasound education perspective this combination of ultrasound image and pressure sensor display can also be used in ultrasound simulation learning to improve ultrasound scanning skill. This invention combines the advantages of an objective palpation device with ultrasound to enhance patient assessment by providing an internal ultrasound image of the structures being palpated and a measure of the probe pressure force being applied. This is a novel approach that will advance ultrasound education, clinical practice, and medical research.

At present ultrasound probes do not have a mechanism to quantitatively measure the degree of contact pressure on the patient's body while being examined. It can be difficult for the sonographer, especially a student or inexperienced sonographer, to appreciate whether the probe pressure is adequate to capture a good image or is excessive and possibly causing unnecessary discomfort or pain to the patient. Having the pressure sensor feedback will improve the scanning process and over time the sonographer can more readily learn the appropriate range of probe pressures. Pain is a common patient complaint for a wide variety of medical conditions, many of which utilize ultrasound in assessing the cause of the pain. Having a pressure sensor ultrasound probe with the ability to determine the pain threshold of the patient and the location of the pain while also visualizing the potential source of the pain with ultrasound beneath the skin offers a tremendous diagnostic advantage and can also serve as a therapeutic management tool. Having an ultrasound probe that can accurately measure and record contact pressure force, especially in a handheld ultrasound device, not only means the pressure data can be used to complement the ultrasound findings, it means it can also be used independent of the ultrasound feature to assess pain in cases when ultrasound imaging is not relevant or possible or the clinician is not trained in ultrasonography. Thus, this medical device can be seen as essentially two diagnostic tools that can be used in combination or separately. This will eliminate the need for having two devices to carry from location to location when seeing patients.

Ultrasound elastography to assess soft tissue in patients is rapidly becoming a standard of medical practice. Elastography is based on changes in the stiffness or compressibility of soft tissue and can indicate pathology. Having an ultrasound probe that has elastography functions as well as the ability to quantitatively measure compressive force would be a major advance in the field of ultrasound elastography improving accuracy, reliability, and diagnostic capabilities of elastography while likely identifying new clinical applications. This invention can be applied to ultrasound simulation education as well as ultrasound robotics. In both of these areas, the amount of pressure applied with the probe whether to a simulation model or a patient robotically is important and pressure measurement is presently not available within the probe itself.

Establishing objective probe pressure measurements to include pain thresholds and elastography effects offer important additional artificial intelligence data points for diagnostic, therapeutic, and personalized medicine advances.

The current disclosure provides multiple novel features including but not limited to: (1) combination of ultrasound and probe contact pressure measurements in a single medical device; (2) quantitative real-time probe pressure as well as color-coded training feedback display for developing a proprioceptive appreciation of probe pressure levels and improvement in ultrasound scanning skill; (3) quantitative probe pressure measurements can be displayed for the entire probe as well as differential measurements for sections of the probe; (4) probe pressure colors can be overlaid on the ultrasound image consistent with the ultrasound wave location on the probe; (5) ability to detect and measure pain in a patient undergoing ultrasound; (6) a pain pressure threshold measurement for the structure beneath the probe can be identified and recorded; (7) combining the additional clinical data point of pain pressure threshold with the ultrasound image to enhance patient diagnosis and management; (8) a combination of ultrasound and probe contact pressure that can be applied to ultrasound simulation training; (9) can be used to enhance and expand the use of ultrasound elastography by providing accurate measures of probe pressure in assessing compressibility and tissue stiffness; (10) the pressure sensor method is built directly into the probe for robotic applications; (11) a device that can be used for pressure/force measurements or ultrasound imaging or the combination of the two; and (12) creation of objective pressure measurements and visual clinical data for use in medical artificial intelligence applications; and (13) application of probe pressure/force measurement and display methodology to all types of ultrasound probes and all forms of probe-display system connectivity including cable connectivity, wireless connectivity, or a combination of connectivity methods.

FIG. 1 shows a traditional linear ultrasound probe 100 with probe surface 102 at (a) not compressed; and (b) compressed.

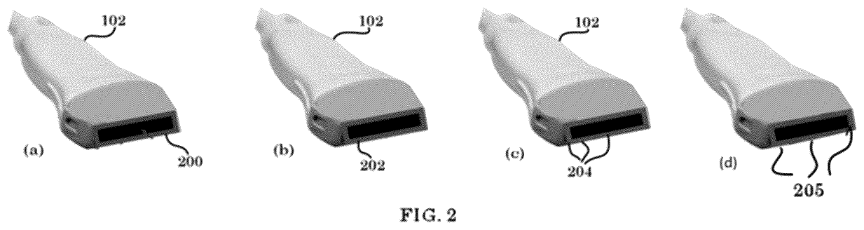

FIG. 2 shows at: (a) traditional linear ultrasound probe 100 with supporting frame 200; (b) a traditional ultrasound probe fitted with a continuous pressure sensor 202; and (c) a traditional ultrasound probe fitted with a pressure sensor array 204 that may be evenly or randomly spaced around supporting frame 200; and (d) a traditional ultrasound probe fitted with pressure sensor sections frame 205. These are examples of potential sensor distributions for a linear ultrasound probe, which can be modified further for other shaped probes such as the curvilinear and sector probes.

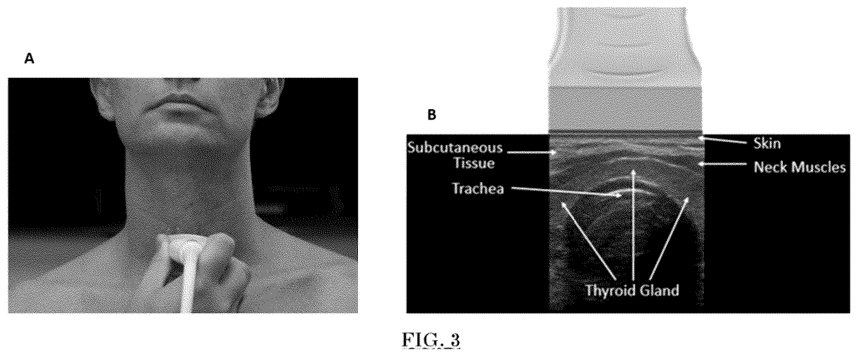

FIG. 3 shows at: (A) a linear probe placed to examine a patient's thyroid gland; and (B) an image of ultrasound images for structures below the skin surface adjacent the ultrasound probe.

FIG. 4 shows at: (A) a linear probe 100 pressure sensor 202 examining the thyroid gland of a patient with pressure sensors visually and numerically, in Newtons (N/cm²), kilograms of force (kgf/cm²), or kilopascal (kPa), displayed showing an evenly distributed moderate (green) probe pressure, reflected in probe pressure display 405. Visual indicators, such as color 402, pressure ranges, etc., may be used to show the distribution of pressure. Here, a color code system of yellow for light pressure, green for moderate pressure, and red for heavy pressure can help the user better place the probe and exert appropriate pressure. FIG. 4 at (B) shows tissue in the probe “path” wherein the image is illustrated 404 in green overlay to show evenly distributed moderate pressure throughout the thyroid and surrounding tissue. The color display does not correlate with the quality of the image automatically. The learner comes to appreciate how much pressure is needed to capture a “good” image at various level and also learns from the heel-toe maneuver.

FIG. 5 shows at: (a) linear probe 100 with the probe sensors displaying a pressure gradient from left to right in the image wherein at 502 the pressure is heavy, moderate pressure 504, and light pressure 508. FIG. 5 at (b) shows color coding of the ultrasound image after activation of this feature with heavy pressure 510, moderate pressure 512, and light pressure 514 displayed as colorations overlaid on the ultrasound image to show the user what pressure is being applied with the probe. The user can adjust these pressures and assess the quality of the image to better appreciate how probe pressure can affect the quality of the image while learning the important heel-toe maneuver of shifting the pressure from heavy (red) at the heel end of the probe to light (yellow) pressure at the toe end of the probe. It should be noted that the pressure color overlay reflects the pressure applied at the point of contact of the probe and the body surface. The applied pressure would only be uniform throughout the depth of the image if the material under the probe is homogeneous throughout.

The current disclosure provides a combination ultrasound and probe contact pressure measurements in a single medical device. It can provide quantitative real-time, probe pressure, as well as color-coded training feedback display for developing a proprioceptive appreciation of probe pressure levels and improvement in ultrasound scanning skill. Indeed, quantitative probe pressure measurements can be displayed for the entire probe as well as differential measurements for sections of the probe. Probe pressure colors can be overlaid on the ultrasound image consistent with the ultrasound wave location on the probe. The current system also provides the ability to detect and measure pain in a patient undergoing ultrasound via the pressure applied to the patent eliciting a pain response. Further, the current disclosure may provide a pain pressure threshold measurement for the structure beneath the probe, which can be identified and recorded. Combining the additional clinical data point of pain pressure threshold with the ultrasound image to enhance patient diagnosis and management. The probes and systems of the current disclosure may also be applied to ultrasound simulation training. Indeed, the current disclosure may be used to enhance and expand the use of ultrasound elastography by providing accurate measures of probe pressure in assessing compressibility and tissue stiffness.

Additionally, the pressure sensor method is built directly into the probe for robotic applications. Robotic ultrasound is the fusion of a robotic system and an ultrasound station with its probe attached to the robot end-effector. This combination might overcome ultrasound disadvantages by means of either a teleoperated, a collaborative assisting, or even an autonomous system. A range of commercial and research systems have been developed over the past two decades for different medical fields, and many were summarized in previous reviews. See, Priester A M, Natarajan S, Culjat M O. Robotic ultrasound systems in medicine. IEEE Trans Ultrason Ferroelectr Freq Control. 2013; 60:507-523. doi: 10.1109/TUFFC.2013.2593 and Swerdlow D R, Cleary K, Wilson E, Azizi-Koutenaei B, Monfaredi R. Robotic arm-assisted sonography: review of technical developments and potential clinical applications. AJR Am J Roentgenol. 2017; 208:733-738. doi: 10.2214/AJR.16.16780.

With their potential for high precision, dexterity, and repeatability, robots are often uniquely suited for ultrasound integration. Although the field is relatively young, it has already generated a multitude of robotic systems for application in dozens of medical procedures. This paper reviews the robotic ultrasound systems that have been developed over the past two decades and describes their potential impact on modern medicine. The robotics ultrasound (RUS) projects reviewed include extracorporeal devices, needle guidance systems, and intraoperative systems. Such systems may include MELODY™ available from ADECHOTECH, 7 rue du Clos Haut de la Bouchardiere 41100 Naveil, France. Indeed, the current disclosure provides a device that can be used for pressure/force measurements or ultrasound imaging or the combination of the two that allows for the creation of objective pressure measurements and visual clinical data for use in medical artificial intelligence applications.

In robotics, using an ultrasound probe with pressure sensors that displays probe pressure measurements on the ultrasound screen as well as color overlay of the degree of pressure on the ultrasound image should allow adjustment of the probe pressure resulting in a higher quality image than pressure/force sensors in the robotic arm/hand alone. In addition, the current disclosure will allow for localization and correlation of probe pressure with any pain the patient may be experiencing from the probe pressure.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. An improved ultrasound probe comprising:
at least one ultrasound probe comprising at least one pressure sensor;
wherein the at least one pressure sensor is configured to measure pressure applied to a patient via the at least one ultrasound probe during ultrasound scanning; and
at least one color-coded feedback display configured to display different colors correlating to different pressure levels.

2. The improved ultrasound probe of claim 1, further comprising configuring the at least one ultrasound probe to display pressure measurements for an entirety of the at least one ultrasound probe.

3. The improved ultrasound probe of claim 1, further comprising configuring the at least one ultrasound probe to display differential measurements for at least one section of the at least one ultrasound probe.

4. The improved ultrasound probe of claim 1, further comprising configuring the at least one ultrasound probe to provide connectivity via cable connectivity, wireless, connectivity, or a combination of connectivity methods.

5. The improved ultrasound probe of claim 1, further comprising a probe contact configured to provide feedback regarding probe pressure applied to the patient.

6. An improved system for conducting ultrasound scans comprising;
at least one ultrasound probe comprising at least one pressure sensor; and
wherein the at least one pressure sensor is configured to measure pressure applied to a patient via the at least one ultrasound probe during ultrasound scanning.

7. The improved system for conducting ultrasound scans of claim 6, further comprising configuring the at least one ultrasound probe to display pressure measurements for an entirety of the at least one ultrasound probe.

8. The improved system for conducting ultrasound scans of claim 6, further comprising configuring the at least one ultrasound probe to display differential measurements for at least one section of the at least one ultrasound probe.

9. The improved system for conducting ultrasound scans of claim 6, further comprising configuring the at least one ultrasound probe to provide connectivity via cable connectivity, wireless, connectivity, or a combination of connectivity methods.

10. The improved system for conducting ultrasound scans of claim 6, further comprising a probe contact configured to provide feedback regarding probe pressure applied to the patient.

11. The improved system for conducting ultrasound scans of claim 6, further comprising at least one color-coded feedback display configured to display different colors correlating to a different pressure levels of the at least one ultrasound probe.

12. An improved ultrasound diagnostic method comprising:
incorporating at least one pressure sensor into at least one ultrasound probe;
wherein the at least one pressure sensor is configured to measure pressure applied to a patient via the at least one ultrasound probe during ultrasound scanning; and
incorporating at least one color-coded feedback display configured to display different colors correlating to different pressure levels.

13. The improved ultrasound diagnostic method of claim 12, further comprising increasing or reducing pressure of the at least one ultrasound probe in response to a pain indication from a patient.

14. The improved ultrasound diagnostic method of claim 12, further comprising increasing or reducing pressure of the at least one ultrasound probe to improve image quality of the at least one ultrasound probe.

15. The improved ultrasound diagnostic method of claim 12, further comprising configuring the at least one ultrasound probe to display pressure measurements for an entirety of the at least one ultrasound probe.

16. The improved ultrasound diagnostic method of claim 12, further comprising configuring the at least one ultrasound probe to display differential measurements for at least one section of the at least one ultrasound probe.

17. The improved ultrasound diagnostic method of claim 12, further comprising configuring the at least one ultrasound probe to provide connectivity via cable connectivity, wireless, connectivity, or a combination of connectivity methods.

18. The improved ultrasound diagnostic method of claim 12, further comprising a probe contact configured to provide feedback regarding probe pressure applied to the patient.
